(19) United States
(12) Patent Application Publication
(10) Pub. No.: US 2012/0301960 A1
(43) Pub. Date: Nov. 29, 2012

(54) Lance Device and Associated Methods for Delivering a Biological Material Into a Cell

(75) Inventors: Quentin T. Aten, Orem, UT (US); Sandra H. Burnett, Saratoga Springs, UT (US); Brian D. Jensen, Orem, UT (US); Larry L. Howell, Orem, UT (US)

(73) Assignee: Brigham Young University, Provo, UT (US)

(21) Appl. No.: 13/480,083

(22) Filed: May 24, 2012

Related U.S. Application Data
(60) Provisional application No. 61/489,548, filed on May 24, 2011.

(51) Int. Cl. C12M 1/00; C12N 5/071

(52) U.S. Cl. 435/375; 435/283.1

(57) ABSTRACT

Systems, devices, and methods for delivering a biological material into a cell are provided. In one example, a lance device for introducing biological material into a cell and configured for use in a nanoinjection system including a microscope is provided. Such a device can include a lance having a tip region and a shaft region, wherein the lance is structurally configured to allow entry and movement of the tip region into the cell along an elongate axis of the tip region and along a focal plane of the microscope. In another example, the lance can be configured to allow substantially horizontal entry and movement of the tip region into the cell.

### PRIORITY DATA

This application claims the benefit of U.S. Provisional Patent Application No. 61/489,548, filed on May 24, 2011, which is incorporated herein by reference.

### BACKGROUND OF THE INVENTION

Microinjection of foreign materials into a biological structure such as a living cell can be problematic. Various transfection techniques include the microinjection of foreign genetic material such as DNA into the nucleus of a cell to facilitate the expression of foreign DNA. For example, when a fertilized oocyte (egg) is transfected, cells arising from that oocyte will carry the foreign genetic material. Thus in one application, organisms can be produced that exhibit additional, enhanced, or repressed genetic traits. In some cases, researchers have used microinjections to create strains of mice that carry a foreign genetic construct causing macrophages to auto-fluoresce and undergo cell death when exposed to a certain drugs. Such transgenic mice have since played roles in investigations of macrophage activity during immune responses and macrophage activity during tumor growth.

Prior art microinjectors function in a similar manner to macro-scale syringes: a pressure differential forces a liquid through a needle and into the cell. In some cases a glass needle that has been fire drawn from a capillary tube can be used to pierce the cellular and nuclear membranes of an oocyte. Precise pumps then cause the expulsion of minute amounts of genetic material from the needle and into the cell. Researchers have produced fine microinjection needles made from silicon nitride and silica glass that are smaller than fire drawn capillaries. These finer needles generally also employ macro-scale pumps similar to those used in traditional microinjectors.

### SUMMARY OF THE INVENTION

The present disclosure provides systems, devices, and methods for delivering a biological material into a cell. In one aspect, for example, a lance device for introducing biological material into a cell and configured for use in a nanoinjection system including a microscope is provided. Such a device can include a lance having a tip region and a shaft region, wherein the lance is structurally configured to allow entry and movement of the tip region into the cell along an elongate axis of the tip region and along a focal plane of the microscope.

In another aspect, a nanoinjection system for introducing biological material into a cell is provided. Such a system can include a lance having a tip region and a shaft region, where the lance is structurally configured to allow entry and movement of the tip region into the cell along an elongate axis of the tip region and along a focal plane of a microscope. The system can also include a charging system electrically coupleable to the lance and being operable to charge and discharge the lance and a lance manipulation system operable to move the lance into and out of a cell in a reciprocating motion along an elongate axis of the lance that minimizes damage to the cell.

In yet another aspect, a method for introducing biological material into a cell is provided. Such a method can include bringing into proximity outside of a cell a lance and a preselected biological material, where the lance having tip region and a shaft region, and charging the lance with a polarity and a charge sufficient to electrically associate the preselected biological material with the tip region. The method can also include moving the lance toward the cell and penetrating a cellular membrane of the cell with the tip region of the lance along an elongate axis of the tip region and along a focal plane of a microscope, and discharging the lance to release at least a portion of the biological material from the tip region. The method can also include withdrawing the lance from the cell.

### BRIEF DESCRIPTION OF THE DRAWINGS

FIG. 1a shows a schematic representation of a step of the delivery of a biological material into a cell in accordance with one embodiment of the present invention.

FIG. 1b shows a schematic representation of a step of the delivery of a biological material into a cell in accordance with another embodiment of the present invention.

FIG. 1c shows a schematic representation of a step of the delivery of a biological material into a cell in accordance with another embodiment of the present invention.

FIG. 1d shows a schematic representation of a step of the delivery of a biological material into a cell in accordance with another embodiment of the present invention.

FIG. 1e shows a schematic representation of a step of the delivery of a biological material into a cell in accordance with another embodiment of the present invention.

FIG. 2 is a graphical representation of a lance in accordance with another embodiment of the present invention.

FIG. 3 is a graphical representation of a lance in accordance with another embodiment of the present invention.

FIG. 4 is a graphical representation of a lance and an injection procedure in accordance with another embodiment of the present invention.

FIG. 5 is a graphical representation of a lance in accordance with another embodiment of the present invention.

FIG. 6a is a graphical representation of a lance in accordance with another embodiment of the present invention.

FIG. 6b is a graphical representation of a lance and an injection procedure in accordance with another embodiment of the present invention.

FIG. 7 is a graphical representation of a lance in accordance with another embodiment of the present invention.

FIG. 8 is a graphical representation of a lance and an injection procedure in accordance with another embodiment of the present invention.

FIG. 9 is a graphical representation of a lance in accordance with another embodiment of the present invention.

FIG. 10 is a graphical representation of a lance and an injection procedure in accordance with another embodiment of the present invention.

### DETAILED DESCRIPTION

In describing and claiming the present invention, the following terminology will be used in accordance with the definitions set forth below.

The singular forms “a,” “an,” and, “the” can include plural referents unless the context clearly dictates otherwise. Thus, for example, reference to “a support” can include reference to one or more of such supports, and reference to “an oocyte” can include reference to one or more of such oocytes.

As used herein, the term “biological material” can refer to any material that has a biological use and can be delivered into a cell or a cell organelle. As such, “biological material” can refer to materials that may or may not have a biological origin. Thus, such material can include natural and synthetic materials, as well as chemical compounds, dyes, and the like.

As used herein, the term “charged biological material” may be used to refer to any biological material that is capable of being attracted to or associated with an electrically charged structure. Accordingly, the term charged biological material may be used to refer to those molecules having a net charge, as well as those molecules that have a net neutral charge but possess a charge distribution that allows attraction to the structure.

As used herein, the term “peptide” may be used to refer to a natural or synthetic molecule comprising two or more amino acids linked by the carboxyl group of one amino acid to the alpha amino group of another. A peptide of the present invention is not limited by length, and thus “peptide” can include polypeptides and proteins.

As used herein, the term “uncharged” when used in reference to a lance may be used to refer to the relative level of charge in the lance as compared to a charged biological material. In other words, a lance may be considered to be “uncharged” as long as the amount of charge on the needle structure is insufficient to associate therewith a useable portion of the charged biological material. Naturally, what is a useable portion may vary depending on the intended use of the biological material, and it should be understood that one of ordinary skill in the art would be aware of what a useable portion is given such an intended use. Additionally it should be noted that a lance with no measurable charge would be considered “uncharged” according to the present definition.

As used herein, “associate” is used to describe biological material that is in electrostatic contact with a structure due to attraction of opposite charges. For example, DNA that has been attracted to a structure by a positive charge is said to be associated or electrically associated with the structure.

As used herein, the term “sample” when used in reference to a sample of a biological material may be used to refer to a portion of biological material that has been purposefully attracted to or associated with the lance. For example, a sample of a biological material such as DNA that is described as being associated with a lance would include DNA that has been purposefully attracted thereto, but would not include DNA that is attracted thereto through the mere exposure of the lance to the environment. One example of DNA that would not be considered to be a “sample” includes airborne DNA fragments that may associate with the lance following exposure to the air.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint without affecting the desired result.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

The present disclosure provides methods, devices, and associated systems for delivering a biological material into a cell with enhanced results. As one non-limiting example, DNA can be delivered into a cell or into an organelle of the cell such as the nucleus or a pronucleus, resulting in genomic integration of the DNA with increased cell and embryo survival rates and increased progeny. While not intending to be bound to any scientific theory, such increased survival rates may be the result of reduced cellular damage from the DNA delivery as compared to prior techniques.

Generally, the present methods, devices, and systems utilize the electrical association and dissociation of a biological material to the lance as a mechanism for delivering the biological material into a biological structure such as a cell or cellular organelle. Because the biological material can be loaded onto the lance and subsequently released via changes in the electrical charge state of the lance, internal microinjection channels are not required for the delivery of the biological material into the cell. As such, a lance can be smaller in size and can be formed in configurations that may not be possible with prior delivery devices. These delivery devices can have an outer shape and cross-section that is significantly smaller than traditional injection pipettes. Such smaller outer shapes may be less disruptive to cellular structures, and thus may allow delivery of the biological material into an organelle with less cellular damage.

Once a biological material has been electrically associated with a tip portion of the lance, the lance can be inserted into a cell. With a tip portion of the lance located within the cell, the lance can be discharged to release at least a portion of the biological material. Once the biological material has been delivered into the cell, the lance can be withdrawn.

Biological material can be delivered into a variety of cells. Both prokaryotic and eukaryotic cells are contemplated that can receive biological material, including cells derived from, without limitation, mammals, plants, insects, fish, birds, yeast, fungus, and the like. Additionally, cells can include somatic cells or germ line cells such as, for example, oocytes and zygotes. The enhanced survivability of cells with the present techniques can allow the use of cells and cell types that have previously been difficult to microinject due to their delicate nature.

Additionally, various types of biological materials are contemplated for delivery into a cell, and any type of biological material that can be electrostatically delivered is considered to be within the present scope. The biological material can be a macromolecule or other material that exists outside of the cell that has been preselected for delivery into the cell. Non-limiting examples of such biological materials can include DNA, cDNA, RNA, siRNA, tRNA, mRNA, microRNA, peptides, synthetic compounds, polymers, dyes, chemical compounds, organic molecules, inorganic molecules, and the like, including combinations thereof. In one aspect, the biological material can include DNA, cDNA, RNA, siRNA, tRNA, mRNA, microRNA, and combinations thereof. In another aspect, the biological material can include DNA and/or cDNA.

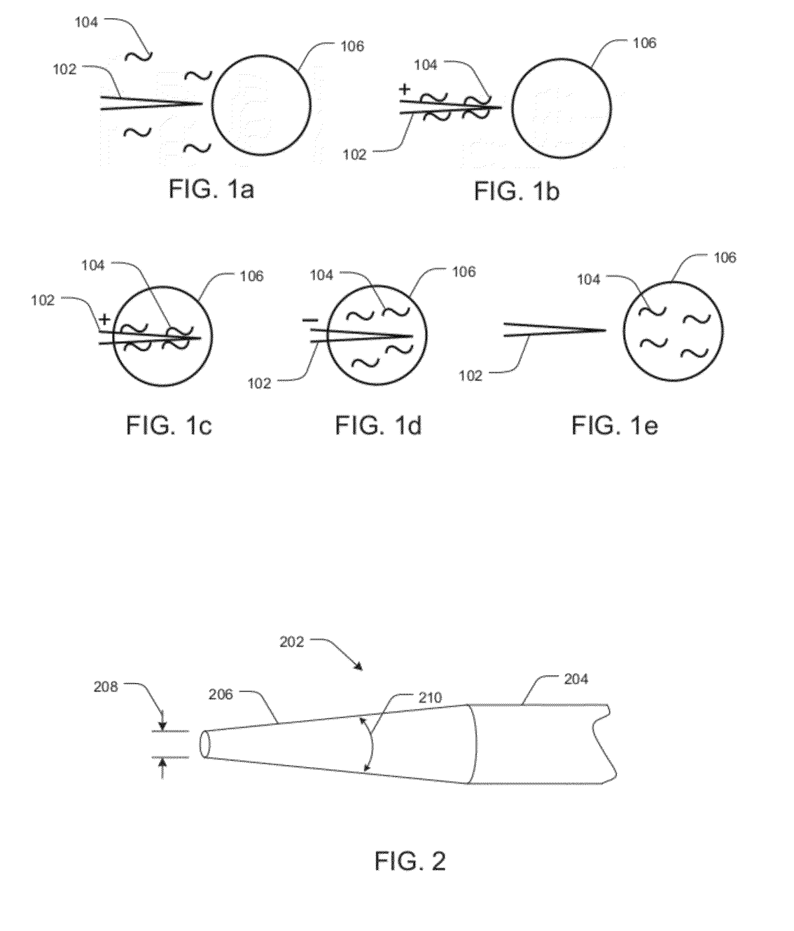

FIGS. 1a-e show an exemplary sequence of steps that can be performed to introduce biological material into a cell according to aspects of the present disclosure. For this particular example, DNA is used as the biological material. This example is intended to be non-limiting, and the description should be applied to other biological materials, cells, organelles, and the like. Electrically-mediated delivery of DNA into an organelle can be accomplished due to the unequal charge distributions within DNA molecules. With an effective charge of 2 electrons per base pair, DNA can be manipulated by an electric field. As is shown in FIG. 1a, a lance 102 and DNA 104 are brought into proximity outside of a cell 106. The DNA 104 can be introduced into proximity of the cell and/or the lance by a biological material delivery device (not shown) to effectively allow the DNA to associate with the tip portion of the lance. Thus, in some aspects it can be beneficial to position the biological material delivery device in sufficient proximity to the lance 102 to facilitate this association. It is contemplated that the biological material delivery device can be physically spaced at any distance from the lance; however diffusion of the biological material may occur upon release, thus lowering the effective concentration of the biological material interacting with the tip portion of the lance. In some aspects it can be beneficial to move the lance through the released biological material (e.g. DNA) in order to further facilitating interaction between the two. It should be noted that the lance can be charged before, after, or during introduction of the biological material into the medium surrounding the lance. Additionally, in some aspects the lance can be introduced into the medium after the introduction of biological material into the medium. Various devices are contemplated, and in one aspect the biological material delivery device can be a micropipette. It is also contemplated that in some aspects the biological material can be distributed homogenously throughout the medium at a concentration that is sufficient to allow a desired amount to accumulate at the tip portion of the lance upon charging.

The polarity of the charge on the lance would depend on the charge distribution of the biological material. In the example shown in FIG. 1, DNA is the biological material and therefore the lance is charged with a positive polarity to associate the DNA molecules thereto. The positive charge on the lance thus causes the negatively charged DNA to associate with and accumulate at the tip portion of the lance. If a biological material having a positive charge distribution is to be delivered, the lance can correspondingly be charged with a negative polarity in order to associate this positively charged biological material to the tip portion of the lance.

As such, the lance 102 is positively charged and brought into contact with the DNA 106 which is accumulated at the tip portion of the lance as is shown in FIG. 1b. The positive charge on the lance 102 thus causes the negatively charged DNA 104 to associate with and accumulate at the tip portion of the lance 102. A return electrode is placed in electrical contact with the medium surrounding the lance in order to complete an electrical circuit with the charging device (not shown). The lance is charged to a degree that is sufficient to associate DNA to the lance during the injection procedure. The amount of voltage sufficient to charge the lance can vary depending on a variety of factors, such as the desired speed of the loading of DNA on the lance, the composition of the lance material, the electrochemical nature of the medium surrounding the lance, and the like.

It should be noted, that various materials begin to decompose (e.g. by electrolysis) at voltages above a certain threshold voltage referred to as the decomposition voltage. The decomposition voltage can be different for different materials. In some cases, such decomposition can generate oxygen and hydrogen at the positively charged lance and the negatively charged return electrode, respectively. These electrolysis products can cause damage to the lance and negatively affect the cell being injected. As such, in one aspect the voltage that can be used to charge the lance can be at or below the decomposition voltage. In one specific aspect, the lance is charged with a voltage from about 1 V below the decomposition voltage to about the decomposition voltage. In another aspect, the lance is charged with a voltage from about 2 V below the decomposition voltage to about the decomposition voltage.

Additionally, voltages higher than the decomposition voltage can cause the biological material to electrophoretically move to the lance. The higher the voltage, the more quickly the biological material will move to and associate with the lance. As such, in some aspects a charging voltage that is higher than the decomposition voltage of the lance can be used. In one aspect, for example, the lance is charged with a voltage from about the decomposition voltage to about 1 V above the decomposition voltage. In another aspect, the lance is charged with a voltage from about the decomposition voltage to about 2 V above the decomposition voltage. In yet another aspect, the lance is charged with a voltage from about the decomposition voltage to about 5 V above the decomposition voltage. In a further aspect, the lance is charged with a voltage that is greater than about 5 V above the decomposition voltage. Additionally, such charging can be described in terms that do not include decomposition voltage. In one aspect, for example, the lance is charged with a voltage from about 0.5 to about 5.0 V. In another aspect, the lance is charged with a voltage from about 1.0 V to about 3 V. In yet another aspect, the lance is charged with a voltage of about 1.5 V.

In some aspects, the cell can be secured by a cell manipulation device to facilitate the injection. The cell can be manipulated, secured, and/or held in position by a variety of mechanisms. It should be noted that any technique, device, or system for manipulating, securing, and/or holding a cell in position is considered to be within the present scope. In one aspect, for example, the cell can be held in position by a suction pipette (not shown). A slight suction at the end of such a pipette can hold a cell for sufficient time to accomplish a biological material delivery procedure into an organelle of the cell. Additionally, supporting arms or other physically restraining structures can be used to hold the cell in position during the delivery procedure. Various configurations for support structures would be readily apparent to one of ordinary skill in the art once in possession of the present disclosure, and such configurations are considered to be within the present scope.

Furthermore, the cell can be manipulated to reorient and/or reposition the cell into a desired position. This can be accomplished by various techniques, and any such technique of manipulation, repositioning, or reorienting is considered to be within the present scope. In the case of the suction pipette, for example, the suction can be repeatedly applied and released to allow the cell to rotate at the tip of the suction pipette. In other aspects, the cell can be rolled along a support surface to facilitate repositioning.

As is shown in FIG. 1c, the lance 102 can be inserted through the cell membrane and into the cell 106. DNA 104 associated with the tip portion is inserted into the cell along with the lance 102. The lance 102 is discharged to allow the release of at least a portion of the DNA 104 from the tip portion of the lance 102, thus delivering the DNA into the cell as is shown in FIG. 1d. Discharging the lance to release the biological material can be accomplished in a variety of ways. In one aspect, for example, discharging the lance can include decreasing the charge on the lance to a degree that is sufficient to release at least a portion of the biological material from the lance. In another aspect, discharging the lance can include releasing the charge on the lance sufficient to release the biological material or at least a portion of the biological material from the lance. In yet another aspect, discharging the lance can include reversing the polarity of the charge on the lance to release at least a portion of the biological material. Such a reversal charges the lance to a polarity that is opposite from the polarity used to accumulate the biological material (e.g. the DNA) on the tip portion of the lance. Thus, a positively charged lance can be reversed to a negative charge to cause a negatively charged biological material such as DNA to be released from the surface of the lance. Thus, depending on the manner in which the lance is discharged, from only a portion to substantially all of the biological material associated with the lance can be released.

Following release of the DNA 104, the lance 102 can be withdrawn from the cell 106 as is shown in FIG. 1e. The DNA 104 delivered to the cell 106 can remain in the cell following withdrawal of the lance 102.

It is contemplated that a lance can be fabricated and utilized in traditional manipulation systems such as micromanipulators and the like. Such manipulation systems will be referred to herein as lance manipulation systems. As such, in some aspects the lance is manufactured as a “stand alone” lance, and is not constrained to a fixed substrate upon which the lance was fabricated. For example, a lance can be manufactured from a precursor material, separated from that material, and coupled to a lance manipulation system. In addition to the lance itself, a coupling mechanism may be required in order to couple the lance to a micromanipulator, depending on the approximate outer diameter of the lance. Furthermore, a charging system and a return electrode can be electrically coupled to the lance to facilitate lance charging and discharging.

Any size and/or shape of lance capable of delivering biological material into a cell is considered to be within the present scope. The size and shape of the lance can also vary depending on the cell receiving the biological material. The effective diameter of the lance, for example, can be sized to maximize survivability of the cell. It should be noted that the term “diameter” is used loosely, as in some cases the cross section of the lance may not be circular. Limits on the minimum diameter of the lance can, in some cases, be a factor of the material from which the lance is made and the manufacturing process used. In one aspect, for example, the lance can have a tip diameter of from about nm to about 3 microns. In another aspect, the lance can have a tip diameter of from about 10 nm to about 2 microns. In another aspect, the lance can have a tip diameter of from about 30 nm to about 1 micron. In a further aspect, the lance can have a tip diameter that is less than or equal to 1 micron. As such, in many cases the tip diameter of the lance can be smaller than the resolving power of current optical microscopes, which is approximately 1 micron.

One exemplary configuration of a lance is shown in FIG. 2. The lance 202 has a shaft portion 204 and a tip portion 206. The tip portion 206 also has a tip diameter 208. In many cases, at least the tip portion 206 can have a taper angle 210. It should be noted that the shaft portion can also be tapered in some lance designs, and thus the distinction between the tip portion and the shaft portion may not be apparent. At least a portion of the shaft can be configured to be coupled to a lance manipulation system such as a micromanipulator (not shown).

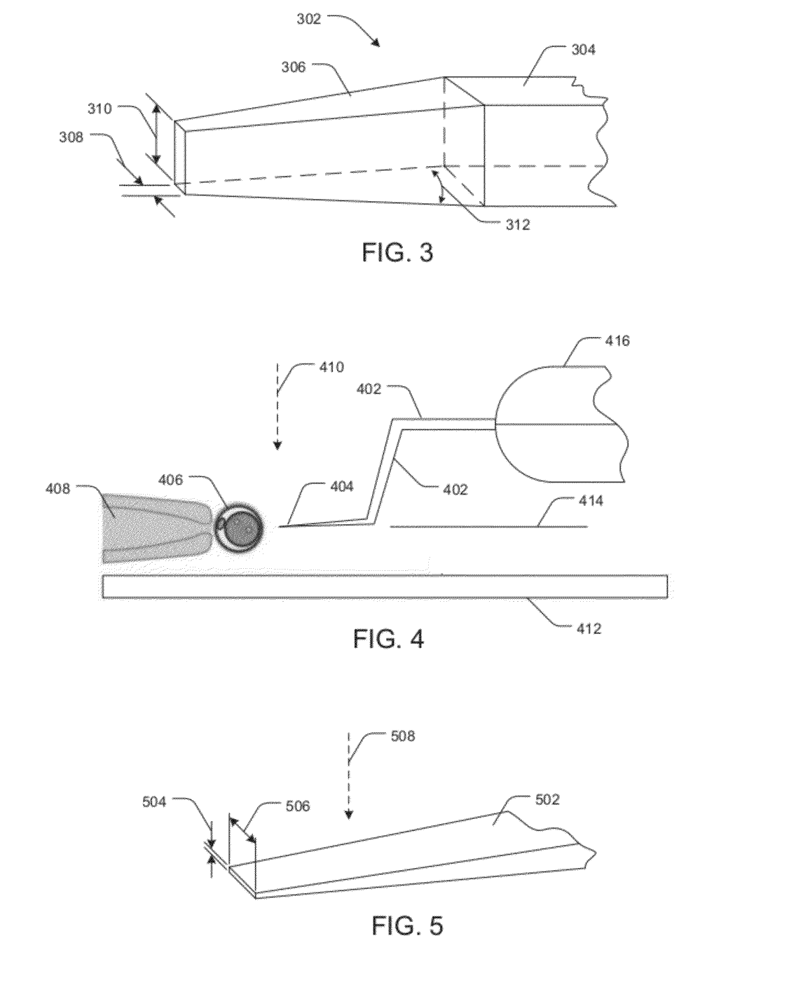

Additionally, lances are contemplated that can have cross sections that are not circular. In such cases, it is intended that the circumference of a circle defined by the tip diameters disclosed above would be substantially the same as an outer circumferential measurement of a non-circular lance tip. One example of such a configuration is shown in FIG. 3. A lance 302 is shown having a shaft portion 304 and a tip portion 306. In this case, the tip portion 306 has a tip thickness 308 and a tip width 310. The lance shown has a taper angle 312. As with the example in FIG. 2, the shaft portion can also be tapered. At least a portion of the shaft can be configured to be coupled to a lance manipulation system such as a micromanipulator (not shown). One non-limiting example of a non-circular lance tip can have a width of from about 0.5 to about 2.0 microns and a thickness of from about 17 to about 200 nanometers.

The delivery of a biological material into a cell can be facilitated by high optical magnification due to the small sizes of such cells. Traditional optical microscopes having sufficient magnification for such delivery are generally oriented with an optical axis in a vertical direction, either coming from above the cell or below the cell for an inverted microscope. When a lance is inserted into a cell, the lance is generally directed toward the cell along an axis that does not correspond to a focal plane of the microscope. A focal plane of the microscope would be perpendicular to the optical axis. If the optical axis is thus oriented in a vertical direction, the focal plane would thus be oriented in a horizontal direction. Because the optics of the microscope are focused at the focal plane and the lance is not oriented along the focal plane, conventional systems have typically required that the lance be continually realigned both horizontally and vertically as it descends toward the cell. Additionally, to facilitate alignment, the microscope is often focused on the tip of the lance, and as such, must be continually refocused as the lance descends toward the cell and out of the current focal plane.

The present disclosure provides the advantage of orienting the lance such that it remains in the focal plane of the microscope as the lance is moved toward the cell. Many manipulation systems preclude such an orientation of the lance due to the proximity of the cell to an underlying substrate and the bulky nature of traditional micromanipulators. The size and physical configurations of the lances according to aspects of the present disclosure, however, allow such in-plane orientation of the lance. As such, in one aspect the present disclosure provides a lance for introducing biological material into a cell and configured for use in a nanoinjection system including a microscope. Such a lance can have a tip region and a shaft region, where the lance is configured to allow entry and movement of the tip region into the cell along an elongate axis of the tip region and along a focal plane of the microscope.

One example of such a configuration is shown in FIG. 4. A lance having a shaft portion 402 and a tip portion 404 is aligned with a cell 406 being held by a pipette 408. The optical axis 410 of the microscope is shown vertically oriented with respect to a support substrate 412, and the focal plane 414 is shown perpendicular to the optical axis 410. As can be seen in FIG. 4, the tip portion 404 is aligned along the focal plane 414. Thus, the tip portion 404 remains in focus within the focal plane 414 as the lance is moved toward and into the cell 406. The lance is shown having a bent configuration that can allow clearance between the lance manipulation system 416 (e.g. micromanipulator) and the support substrate 412.

The tip portion 404 is shown having a horizontal orientation that is in the focal plane 414. However, in various aspects it is contemplated that the focal plane and thus the tip portion of the lance can be in an orientational configuration that is not horizontal, but wherein the elongate axis of the tip portion of the lance is aligned within the focal plane. Thus, it is contemplated that that in some aspects the lance can be used at shallow angles for injections into a cell. In one aspect, for example, a shallow angle can be less than about 30° from the focal plane (or from horizontal). In another aspect, a shallow angle can be less than about 20° from the focal plane (or from horizontal). In yet another aspect, a shallow angle can be less than about 10° from the focal plane (or from horizontal). In a further aspect, a shallow angle can be less than about 5° from the focal plane (or from horizontal). In yet a further aspect, a shallow angle can be less than about 1° from the focal plane (or from horizontal).

Additionally, as has been noted, the resolving power of current optical microscopes is about 1 micron. As such, it can be difficult to optically visualize objects that are smaller than 1 micron. Various lance designs can be utilized that allow small tip sizes that reduce damage done to the cell and that are capable of optical visualization during delivery procedures. In one aspect, for example, a lance for use in a nanoinjection system can have a nanometer-sized tip region that is viewable in an optical microscope. Such a lance can have a tip region having a width that is greater than or equal to about 1 μm and a thickness that is in the nanometer range. Thus the lance tip is readily inserted into a cell due to a nanometer scale thickness. The tip is readily discriminated by optical microscopy because the lance is oriented such that the width is parallel to and thus within the focal plane of the microscope. In one aspect, the thickness can be from about 5 nm to about 500 nm. In another aspect, the thickness can be from about 20 nm to about 200 nm. In yet another aspect, the thickness can be from about 50 nm to about 100 nm. In another aspect, the thickness can be from about 25 nm to about 50 nm. As is shown in FIG. 5, for example, such a lance 502 includes a thickness 504 and a width 506. The lance tip is oriented such that the width 506 is viewable in the focal plane (not shown) of the microscope, which is perpendicular to the optical axis 508.

The length of the lance can be variable depending on the design and desired attachment of the lance to the lance manipulation system. Also, the portion of the lance that is contacting and/or passing through a portion of the cell can vary in length depending on the lance design and the depth of the area into which the biological material is to be delivered. For example, delivering biological material to an area located near the surface of a cell can be accomplished using a shorter lance as compared to delivery to an area located deep within the cell. This would not preclude, however, the use of longer lances for delivery into areas near the cellular surface. For example, a relatively long lance may be used to deliver biological material in an application where only a small portion (e.g., only the tip) of the lance penetrates a cell. It should be noted that the lance length can be tailored to the delivery situation and to the preference of the individual performing the delivery.

Thus the length of the lance can be any length useful for a given delivery operation. For example, in some aspects, the lance can be up to many centimeters in length. In other aspects, the lance can be from a millimeter to a centimeter in length. In another aspect, the lance can be from a micron to a millimeter in length. In one specific aspect, the lance can be from about 2 microns to about 500 microns in length. In another specific aspect, the lance can be from about 2 microns to about 200 microns in length. In yet another specific aspect, the lance can be from about 10 microns to about 75 microns in length. In a further specific aspect, the lance can be from about 40 microns to about 60 microns in length.

Additionally, the shape of the lance, at least through the portion of the lance contacting the cell, can vary depending on the design of the lance and the depth to which the biological material is to be injected into the cell. A high lance taper, for example, may be more disruptive to cellular membranes and internal cellular structures than a low taper. In one aspect, for example, the lance can have a taper of from about 1% to about 10%. In another aspect, the taper can be from about 2% to about 6%. In yet another aspect, the taper can be about 3%. The taper of the lance can also be described in terms of the size of the disruption in the cell membrane following insertion. In one aspect, for example, the approximate diameter of the disrupted area of the cell membrane following lance insertion is from about 0.5 nanometers to about 8 microns. In another aspect, the approximate diameter of the disrupted area of the cell membrane following lance insertion is from about 2 micron to about 5 microns.

The overall shape and size of the lance can also be designed to take into account various factors, including those involved with the delivery procedure, as well as the materials utilized to make the lance. For example, in one aspect a lance can be designed having sufficient cross sectional strength to allow biological material delivery, while at the same time minimizing the damage done to the cell from the lance's cross sectional area. As another example, the lance can be designed to have a cross sectional area sufficient to minimize damage to the cell, while at the same having sufficient surface area to which biological material can be associated.

Different materials can also affect the design of the size and shape of the lance. Some materials may not hold a charge sufficient to associate the biological material to the lance tip at smaller sizes. In such cases, larger size lances can be used to facilitate a higher charge capacity. It may be difficult to form particular sizes and shapes of the lance from certain materials. In such cases, the lance size and shape can be designed to the properties of the desired material. For example, a material such as gold may not be capable of supporting the lance tip at very small diameters due to inadequate strength at smaller sizes, or it may not be possible or feasible to create a very small diameter tip with gold. If the use of a gold lance is desired, the lance size and shape can thus be designed with the properties of gold in mind.

As has been described, a charge is introduced into and held by the lance in order to electrically associate the biological material to the lance. Various lance materials are contemplated for use in constructing the lance, and any material that can be formed into a lance structure and is capable of carrying a charge is considered to be within the present scope. Non-limiting examples of lance materials can include a metal or metal alloy, a conductive glass, a polymeric material, a semiconductor material, carbon nanotube, and the like, including combinations thereof. In one aspect, a lance can be a carbon nanotube filled with a material such as carbon, silicon, and the like. Non-limiting examples of metals can include indium, gold, platinum, silver, copper, palladium, tungsten, aluminum, titanium, and the like, including alloys and combinations thereof. Polymeric materials that can be used to construct the needle structure can include any conductive polymer, non-limiting examples of which include polypyrrole doped with dodecyl benzene sulfonate ions, SU-8 polymer with embedded metallic particles, and the like, including combinations thereof. Non-limiting examples of useful semiconductor materials can include germanium, gallium arsenide, and silicon, including various forms of silicon such as amorphous silicon, monocrystalline silicon, polycrystalline silicon, and the like, including combinations thereof. Indium-tin oxide is a material that is also contemplated for use as a lance material.

Additionally, in some aspects the lance can be a conductive material that is coated on a second material, where the second material provides the physical structure of the lance. Examples can include metal-coated glass or metal-coated quartz lances. The lance can also include a hollow, non-conductive material, such as a glass, where the hollow material is filled with a conductive material. Depending on the design, the lance can be manufactured using various techniques such as wire pulling, chemical etching, MEMs processing, various deposition techniques, and the like.

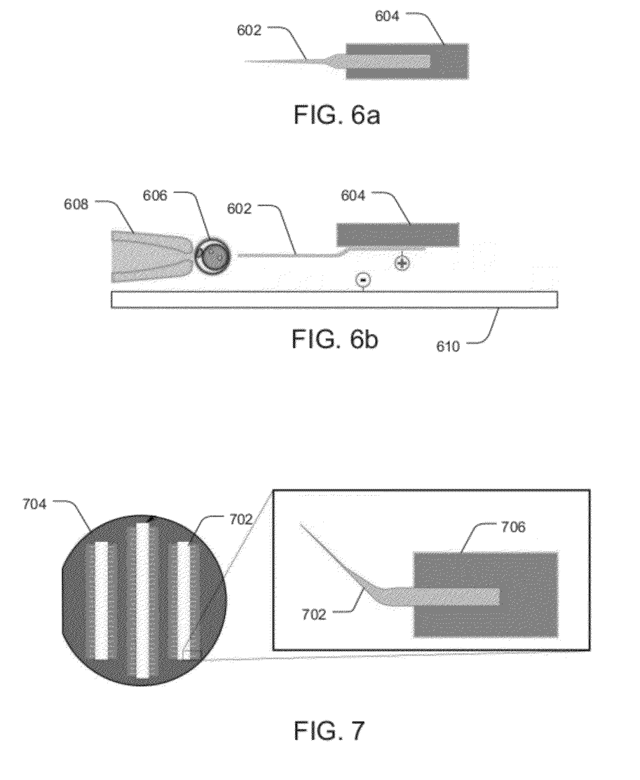

In one aspect, a lance can be fabricated using MEMS processing from semiconductive or other MEMS capable materials. As is shown in FIG. 6a, for example, a polysilicon lance 602 is made in conjunction with a silicon substrate 604, where the silicon substrate 604 can be used to couple the lance to a lance manipulation system. The silicon substrate can also allow electrical connection between a charging system and the polysilicon lance. Accordingly, very small tip sizes can be manufactured in the lance by using such processes. FIG. 6b shows a side view of the polysilicon lance 602 and the silicon substrate 604 aligned to penetrate into a cell 606 held by a cellular manipulation device 608. In this case, a support substrate 610 is utilized as a return electrode. It should be noted that polysilicon and silicon are merely exemplary, and any material that can be formed into such a lance can be utilized.

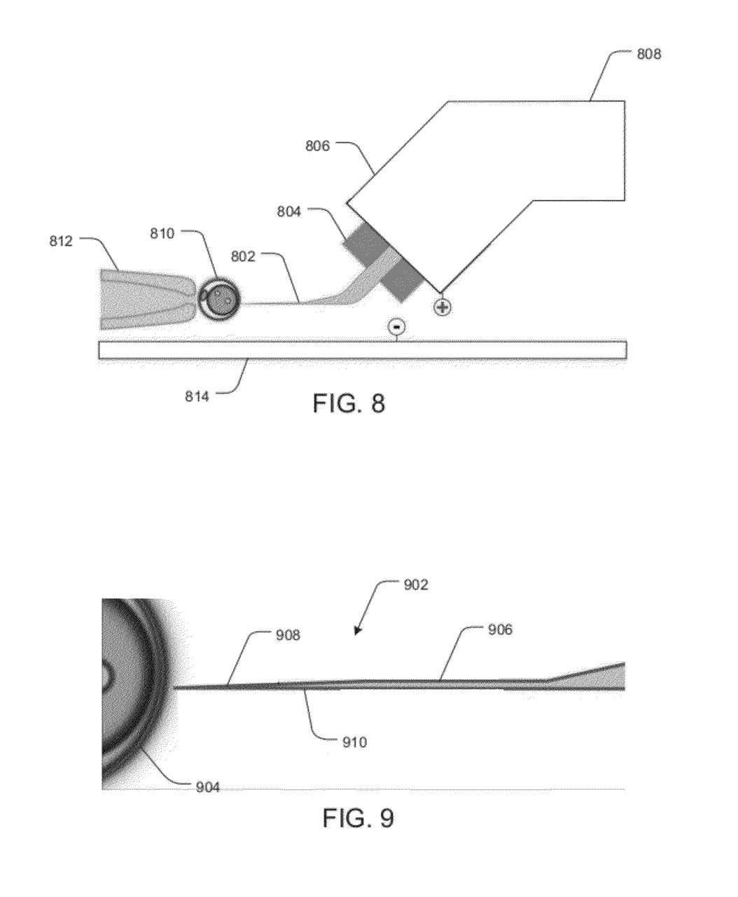

FIG. 7 shows an exemplary embodiment of a plurality of polycrystalline silicon lances 702 manufactured on a silicon wafer 704. The enlarged portion of FIG. 7 shows an individual lance 702 and a silicon substrate 706 that can be used to couple the lance 702 to a lance manipulation system (not shown). In one aspect, the lance can be bent into any configuration that allows movement of the lance in the focal plane of the microscope. Such a lance 802 orientation is also shown in FIG. 8, where the silicon substrate 804 is coupled into a socket of a coupling 806 that is configured at one end 808 to couple with a lance manipulation system (not shown). The physical configuration of the coupling should not be limited to what is shown in FIG. 8, and it should be understood that any other physical configuration is considered to be within the present scope. As such, it is generally contemplated that the coupling can be of any size or configuration that allows attachment between the lance and the lance manipulation system. FIG. 8 also shows a cell 810, a cell manipulation system 812, and a support substrate 814.

FIG. 9 shows a close up view of one exemplary embodiment of a lance tip 902 that has a configuration to allow the lance to remain in the focal plane and the lance is moved toward the cell 904. The lance tip can include a substantially non-tapered region 906 having a substantially constant width along a region penetrating the cell 904. Additionally, the lance tip 902 can include a tapered region 908 at the tip and a substantially flat side 910 that allows the lance tip 902 to remain in focus as it moves along the focal plane into the cell 904. It is also noted that this configuration is merely exemplary, and should not be seen as limiting.

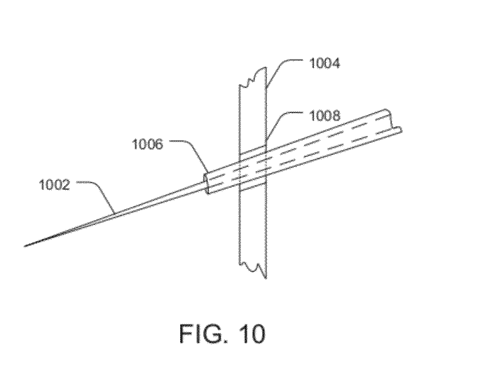

It is also contemplated that the lance can include a passivation layer to focus electrical current and to protect the cellular membrane and other cellular structures from the electrical current. As is shown in FIG. 10, for example, a lance 1002 is shown passing through a cellular membrane 1004. The lance 1002 has a passivation layer 1006 coated onto at least a portion of the lance 1002, but at least a portion of the lance tip is free of the passivation layer 1006. When the lance 1002 is passed through the cellular membrane 1004 to a point where the passivation layer 1006 contacts the cellular membrane 1004, electrical current is focused primarily through the exposed tip of the lance, which in some cases can protect regions of cellular membrane around the injection site 1008 from electrical current damage.

In addition to a protective function, the passivation layer can control the location, magnitude, and/or direction of the electric field and current flow from the tip of the lance. The passivation material can be any material that is less conductive than the material of the lance, and can thus focus current flow and/or protect cellular structures. In one aspect, for example, the passivation material can be any material capable of providing the functionality described. In another aspect, the passivation material can be electrically insulating. Non-limiting examples of passivation materials can include oxides, nitrides, oxynitrides, ceramics, polymers, and the like, including combinations thereof.

A charging system used to charge the lance can include any system capable of electrically charging, maintaining the charge, and subsequently discharging the lance. Non-limiting examples can include batteries, DC power supplies, photovoltaic cells, static electricity generators, capacitors, and the like. The charging system can include a switch for activation and deactivation, and in some aspects can also include a polarity switch to reverse polarity of the charge on the lance. In one aspect the system may additionally include multiple charging systems, one system for charging the lance with a charge, and another charging system for charging the lance with an opposite polarity charge. In one example scenario, an initially uncharged lance is brought into contact with a sample of a biological material. The biological material can be in water, saline, or any other liquid capable of maintaining biological material. A charge opposite in polarity to the biological material is applied to the lance, thus associating a portion of the biological material with the lance. The lance can then be moved into the organelle of interest, and lance can be discharged, thus releasing the biological material.

The lance can be manipulated by any mechanism capable of aligning and moving the lance. Such a lance manipulation system can include any system or device capable of orienting and moving a lance. Non-limiting examples of lance manipulation systems include mechanical systems, magnetic systems, piezoelectric systems, electrostatic systems, thermo-mechanical systems, pneumatic systems, hydraulic systems, and the like. In one aspect, the lance manipulation system can be one or more micromanipulators. The lance may also be moved manually by a user. For example, a user may push the lance along a track from first location to a second location.

In one aspect, the lance can be moved by the lance manipulation system in a reciprocal motion along an elongate axis of the lance. In other words, the lance can move forward into a cell and backward out of the cell along the same path. By moving along the elongate axis of the lance, the minimum cross sectional area of the lance is driven through cellular structures such as a cell membrane and/or a cellular organelle. This minimal cross sectional exposure can limit the cellular disruption, and thus potentially increasing the success of the biological material delivery procedure.

The cell can be manipulated and or held in position by a variety of mechanisms. It should be noted that any technique, device, or system for manipulating and/or holding a cell in position is considered to be within the present scope. In one aspect, for example, the cell can be held in position by a suction pipette. A slight suction at the end of such a pipette can hold a cell for sufficient time to accomplish a biological material delivery procedure into an organelle of the cell. Additionally, supporting arms or other physically restraining structures can be used to hold the cell in position during the delivery procedure. Various configurations for support structures would be readily apparent to one of ordinary skill in the art once in possession of the present disclosure, and such configurations are considered to be within the present scope.

Further exemplary details regarding lances, charging systems, lance manipulation systems, and cellular restraining systems can be found in U.S. patent application Ser. Nos. 12/668,369, filed Sep. 2, 2010; 12/816,183; filed Jun. 15, 2010; 61/380,612, filed Sep. 7, 2010; and 61/479,777, filed on Apr. 27, 2011, each of which is incorporated herein by reference.

The design of a system for delivering biological material into a cell can vary due to the interdependencies of various system parameters. Combinations of features can thus influence other features, both in terms of system design and in terms of system use. Features can thus be mixed and matched to create a delivery system for a given purpose or desirable performance. For example, the materials and configuration chosen for the lance may have properties allowing a greater or lesser charge capacity, thus influencing the voltage, current, and electrical timing of the charging and discharging. A smaller tip diameter can more effectively enter a cell or an organelle with potentially less damage, but may have a smaller surface area for the association of biological material. The association capacity of the lance for biological material can thus be increased, for example, by utilizing lance materials capable of holding a higher relative charge, or by utilizing a non-circular shape for the lance tip that increases surface area while minimizing the penetration damage of the lance. Thus, if a particular feature is desired for a lance, other features can be varied to accommodate such a design. As such, it should be understood that the various details described herein should not be seen as limiting, particularly those involving dimensions or values. It is contemplated that a wide variety of design choices are possible, and each are considered to be within the present scope.

It is to be understood that the above-described compositions and modes of application are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

1. A lance device for introducing biological material into a cell and configured for use in a nanoinjection system including a microscope, comprising:
a lance having a tip region and a shaft region, wherein the lance is structurally configured to allow entry and movement of the tip region into the cell along an elongate axis of the tip region and along a focal plane of the microscope.

2. The lance of claim 1, wherein the lance is structurally configured to be removeably coupled to a micromanipulator device.

3. The lance of claim 1, wherein the lance is structurally configured to allow substantially horizontal entry and movement of the tip region into the cell.

4. The lance of claim 3, wherein the lance is structurally configured such that the tip region remains in a focal plane of the microscope as the lance is moved substantially horizontally into the cell along the elongate axis of the tip region.

5. The lance of claim 3, wherein the lance has a nanometer-sized tip region that is viewable in an optical microscope.

6. The lance of claim 5, wherein the tip region of the lance has a thickness that is greater than or equal to 1 μm and a width that is less than or equal to 500 nm, wherein the lance is structurally configured to have the tip region oriented such that the thickness is viewable in a focal plane of the microscope when the lance is inserted into the cell.

7. The lance of claim 1, further comprising a passivation layer coated on at least a portion of the shaft region of the lance, wherein at least a portion of the tip region of the lance is free of the passivation layer.

8. A nanoinjection system for introducing biological material into a cell, comprising:
a lance having a tip region and a shaft region, wherein the lance is structurally configured to allow entry and movement of the tip region into the cell along an elongate axis of the tip region and along a focal plane of a microscope;
a charging system electrically coupleable to the lance and being operable to charge and discharge the lance; and
a lance manipulation system operable to move the lance into and out of a cell in a reciprocating motion along an elongate axis of the lance that minimizes damage to the cell.

9. The system of claim 8, further comprising a microscope oriented such that a focal plane of the microscope is parallel to the elongate axis of the tip region.

10. The system of claim 8, further comprising a biological material delivery device configured to deliver a biological material capable of association with the lance.

11. The system of claim 10, wherein the biological material delivery device is positioned to deliver the biological material to contact the lance.

12. The system of claim 8, further comprising a preselected biological material sample electrically associated with a tip portion of the lance.

13. The system of claim 8, further comprising a single cell positioned to receive the lance upon operation of the lance manipulation system.

14. The system of claim 13, wherein the single cell is a zygote.

15. A method for introducing biological material into a cell, comprising:
bringing into proximity outside of a cell a lance and a preselected biological material, the lance having tip region and a shaft region;
charging the lance with a polarity and a charge sufficient to electrically associate the preselected biological material with the tip region;
moving the lance toward the cell and penetrating a cellular membrane of the cell with the tip region of the lance along an elongate axis of the tip region and along a focal plane of a microscope;
discharging the lance to release at least a portion of the biological material from the tip region; and
withdrawing the lance from the cell.

16. The method of claim 15, further comprising manipulating the cell to orient the cell into a desired position prior to penetrating the cellular membrane.

17. The method of claim 15, further comprising securing the cell prior to penetrating the cellular membrane and releasing the cell following withdrawing the lance from the cell.
